(19) United States
(12) Patent Application Publication
(10) Pub. No.: US 2011/0098799 A1
(43) Pub. Date: Apr. 28, 2011

(54) Stent Combined with a Biological Scaffold Seeded With Endothelial Cells

(75) Inventors: Kevin Treacy, Galway (IE); Lucy O'Keeffe, Galway (IE); Patrick Duane, Galway (IE); Paul Kenna, Wicklow (IE); Tony O'Halloran, Galway (IE)

(73) Assignee: Medtronic Vascular, Inc., Santa Rosa, CA (US)

(21) Appl. No.: 12/606,789

(22) Filed: Oct. 27, 2009

(51) Int. Cl. A61F 2/06

(52) U.S. Cl. 623/1.13; 623/1.41; 623/1.42

(57) ABSTRACT

Disclosed herein are implantable medical device, and in particular, vascular stent having a biocompatible scaffold seeded with endothelial cells. The stent can provide structural support to maintain the openness of a vessel lumen following angioplasty while the biological scaffold seeded with endothelial cells can provide a new, healthy blood vessel wall.

### FIELD OF THE INVENTION

The present invention relates to an implantable medical device, and in particular, to a vascular stent having a biocompatible scaffold seeded with endothelial cells.

### BACKGROUND OF THE INVENTION

Cardiovascular disease, including atherosclerosis, is the leading cause of death in the United States. The medical community has developed a number of methods and devices for treating atherosclerosis and other forms of coronary arterial narrowing.

One method for treating atherosclerosis is percutaneous transluminal coronary angioplasty, commonly referred to as “angioplasty” or “PTCA”. The objective of angioplasty is to enlarge the lumen of the affected coronary artery by radial hydraulic expansion. The procedure is accomplished by inflating a balloon within the narrowed lumen of the coronary artery. Radial expansion of the coronary artery occurs in several different dimensions, and is related to the nature of the plaque. Soft, fatty plaque deposits are flattened by the balloon, while hardened deposits are cracked and split to enlarge the lumen. The wall of the artery itself is also stretched when the balloon is inflated.

Unfortunately, while affected arteries can be enlarged in this manner, in some instances the vessel restenoses chronically, or closes down acutely, negating the positive effect of the angioplasty procedure. In the past, such restenosis necessitated repeat angioplasty or open heart surgery.

Various devices have been proposed to lessen the risk of restenosis following angioplasty. Stents, one such type of device, are typically inserted into the vessel, positioned across the lesion or stenosis, and then expanded to keep the passageway clear. The stent overcomes the natural tendency of the vessel walls of some patients to restenose, thus maintaining the patency of the vessel.

Typically stents consist of an expansible mesh which is collapsible during insertion into a vessel and thereafter expansible to firmly engage the inner wall surface of a blood vessel and secure it in place. In addition to providing structural support, some stents have been coated with various medications for purposes such as minimizing inflammation and providing treatment. While stents coated with therapeutic agents address many drawbacks associated with angioplasty procedures, there is still room for improvement in providing stents with improved properties.

### SUMMARY OF THE INVENTION

The present invention provides a stent with a biological scaffold seeded with endothelial cells. The stent provides structural support to maintain the openness of the vessel lumen following angioplasty while the biological scaffold seeded with endothelial cells provides a new, healthy blood vessel wall. That is, the endothelial cells act as a non-diseased inner endothelial lumen. This functional endothelium can provide a continuous thromboresistant layer between blood and the blood vessel wall, can control blood flow and vessel tone, platelet activation, adhesion and aggregation, smooth muscle cell migration and proliferation, and can also reduce the disruption of blood flow caused by conventional stents. Such a cell lining also prevents the conversion of fibrin to fibrinogen.

One particular embodiment includes a stent comprising a biological scaffold seeded with endothelial cells on the inner surfaces of the stent. In another embodiment, the biological scaffold is also on the outer surfaces of the stent.

In another embodiment, the biological scaffold comprises extracellular matrix material, submucosa, dura mater, pericardium, serosa, peritoneum and/or a basement membrane tissue. In another embodiment, the submucosa comprises intestinal submucosa, stomach submucosa, urinary bladder submucosa and/or uterine submucosa. In another embodiment, the submucosa comprises at least one growth factor. In another embodiment, the at least one growth factor is basic fibroblast growth factor, transforming growth factor beta, epidermal growth factor and/or platelet derived growth factor.

In another embodiment, the basement membrane tissue comprises liver basement membrane tissue.

In another embodiment, the biological scaffold is derived from a warm-blooded vertebrate.

In another embodiment, the biological scaffold comprises a therapeutic agent.

In another embodiment, the endothelial cells are arterial and/or venous vascular endothelial cells. In another embodiment, the endothelial cells are genetically engineered to express a biologically active protein product.

In another embodiment, the biological scaffold seeded with endothelial cells is about 1 to about 3 cells thick.

Embodiments disclosed herein also include methods. One particular embodiment provides a method of forming a stent comprising a biological scaffold seeded with endothelial cells comprising: providing a stent; seeding a biological scaffold with endothelial cells; and associating the biological scaffold seeded with the endothelial cells with the stent.

Another embodiment disclosed herein includes treating the biological scaffold with glutaraldehyde, formaldehyde, oxidizing compounds, gas plasma sterilization and/or gamma radiation. In another embodiment, the oxidizing compound is a peracid diluted in alcohol. In another embodiment, the peracid is peracetic acid, perpropionic acid or prebenzoic acid and the alcohol is ethanol, propanol, isopropanol, dentatured alcohol or butanol.

Another embodiment disclosed herein includes pre-rinsing the biological scaffold with a sterile solvent before the treating.

In another embodiment, the biological scaffold is submucosa and the method further comprises: processing the submucosa to retain at least one growth factor. In another embodiment, the at least one growth factor is basic fibroblast growth factor, transforming growth factor beta, epidermal growth factor and/or platelet derived growth factor.

Another embodiment disclosed herein includes genetically engineering the endothelial cells to express a biologically active protein product.

### BRIEF DESCRIPTION OF THE FIGURES

FIGS. 1A and 1B illustrate a stent that can be used with embodiments disclosed herein;

FIG. 2 illustrates another stent that can be used with embodiments disclosed herein;

FIG. 3 illustrates yet another stent that can be used with embodiments disclosed herein;

FIG. 4 illustrates a cross-sectional view of a stent strut showing a biological scaffold seeded with endothelial cells in accordance with an embodiment disclosed herein;

FIG. 5 illustrates a stent coated with a biological scaffold seeded with endothelial cells;

FIG. 6 illustrates a delivery catheter used to implant a stent of an embodiment disclosed herein;

FIG. 7 illustrates the stent of FIG. 5 inserted into an occluded artery with an angioplasty balloon in position within the stent and before expansion of the balloon;

FIG. 8 illustrates the stent of FIG. 5 implanted and after the angioplasty balloon has been removed.

### DETAILED DESCRIPTION OF THE INVENTION

Cardiovascular disease, including atherosclerosis, is the leading cause of death in the United States. One method for treating atherosclerosis is percutaneous transluminal coronary angioplasty (“PTCA”), commonly referred to as “angioplasty.” The objective of angioplasty is to enlarge the lumen of the affected coronary artery. Unfortunately, while affected arteries can be enlarged in this manner, they can, in some instances, restenose chronically, or close down acutely, negating the positive effect of the angioplasty procedure.

To address this issue, following angioplasty, stents are often positioned across the stenosis, and expanded to keep the passageway clear. In addition to providing structural support, stents have also been coated with various medications to minimize inflammation and provide treatment. Embodiments disclosed herein provide a stent with a biological scaffold seeded with endothelial cells. The stent provides structural support to maintain the openness of the vessel following the angioplasty procedure while the biological scaffold seeded with endothelial cells provides a new, healthy vessel wall. This functional endothelium can provide a continuous thromboresistant layer between blood and the blood vessel wall, can control blood flow and vessel tone, platelet activation, adhesion and aggregation, smooth muscle cell migration and proliferation, and also reduces the disruption of blood flow caused by conventional stents. Such a cell lining also prevents the conversion of fibrin to fibrinogen.

### Stents

A stent is typically an open mesh cylindrical device that is implanted at an angioplasty site. Stents are generally constructed from materials such as, without limitation, stainless steel (e.g. 316-L stainless steel or 316L5), MP35 alloy, nitinol, tantalum, ceramic, nickel, titanium, aluminum, polymeric materials, tantalum, MP35N, titanium ASTM F63-83 Grade 1, niobium, gold, high carat gold K 19-22, nitinol, platinum, inconel, iridium, silver, tungsten, a biocompatible metal, carbon, carbon fiber or combinations thereof.

Stents of various forms and methods of fabrication can be used in accordance with the embodiments described herein. For example, in a typical method of making a stent, a thin-walled, small diameter metallic tube is cut to produce the desired stent pattern, using methods such as laser cutting or chemical etching. The cut stent may then be descaled, polished, cleaned and rinsed. Stents can also be welded, molded or can consist of various filaments or fibers wound or braided together. Additional non-limiting examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety for methods of forming stents and appropriate stent structures.

Stents can be classified according to whether they are balloon-expandable or self-expanding. Typically, balloon expandable stents are made of stainless steel while self-expanding stents are composed of “smart metals” incorporating shape memory alloys containing nickel and titanium (Nitinol). Shape Memory Alloys (SMAs) refer to alloys that retain their original shape when exposed to a certain temperature threshold. These stents are designed to contract or contort under a cold environment and expand or return to their original shape under warmer temperatures. Stents using SMAs generally contain about 55% nickel and 45% titanium and expand automatically at a body temperature of 37° C. Self-expanding stents often require a suitable coating because nickel can be toxic and can leach out over a prolonged period of time. Both types of stents can be used in the embodiments disclosed herein.

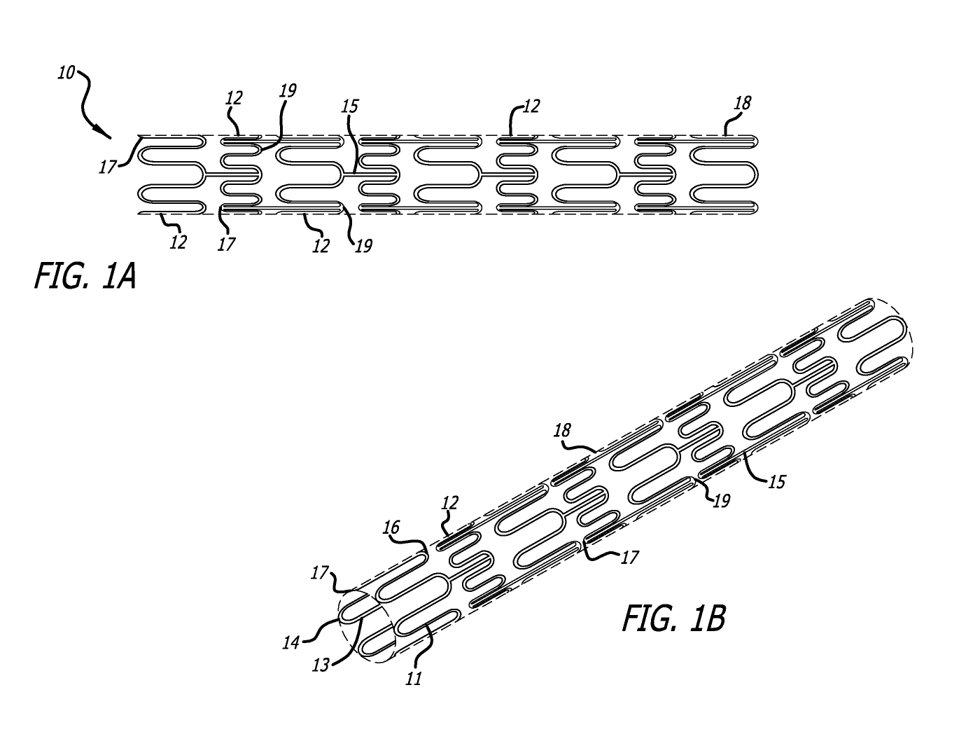

FIGS. 1A and 1B show one stent type appropriate for use with embodiments disclosed herein. Stent 10 is made up of a plurality of cylindrical rings 12 having a strut pattern. The cylindrical rings 12 extend circumferentially around the stent 10 when it is in a tubular form and are coaxially aligned along a common longitudinal axis which forms the stent 10. Each cylindrical ring 12 has a first end 14 (e.g., proximal end) and a second end 16 (e.g., distal end) with the distance between the first, proximal end and the second, distal end defining a ring length.

Each cylindrical ring 12 defines a cylindrical plane 18 and includes a cylindrical outer wall surface 11 which defines the outermost surface of the stent, and a cylindrical inner wall surface 13 which defines the innermost surface of the stent. The cylindrical plane 18 follows the cylindrical outer wall surface and the links 15 are positioned within the cylindrical plane 18. The links 15 couple one cylindrical ring 12 to an adjacent cylindrical ring 12.

This stent 10 can be described more particularly as having peaks 17 and valleys 19 with struts positioned therebetween. The number of peaks and valleys, sometimes referred to as crowns, can vary in number for each ring, in one embodiment, depending on the stent's intended application.

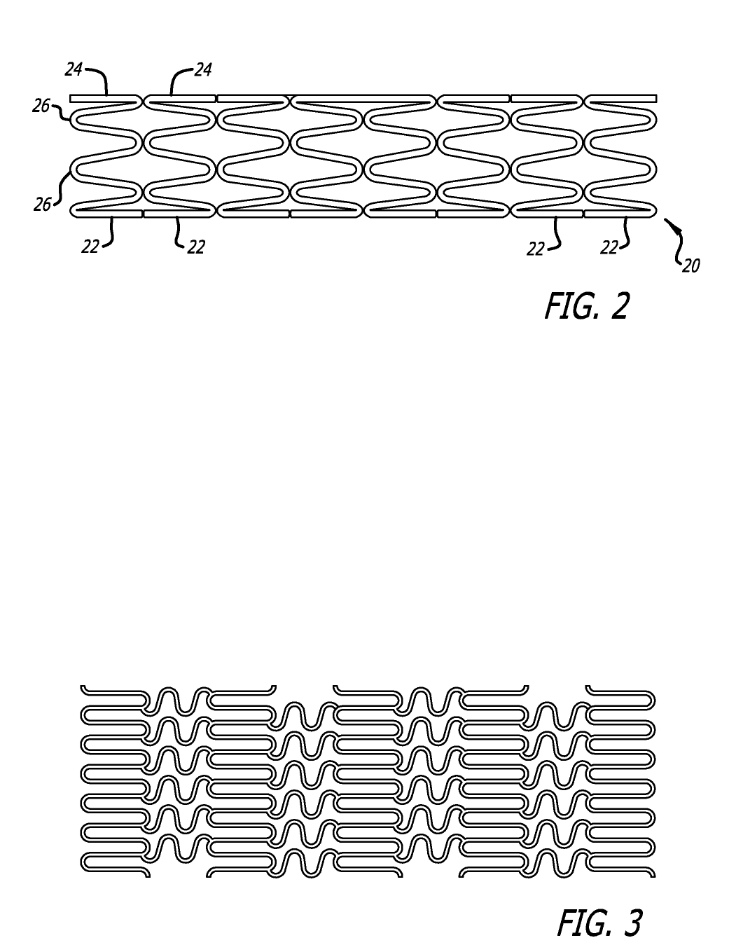

FIG. 2 is a side view of an illustrative embodiment of another stent 20 appropriate for use with embodiments disclosed herein. This stent 20 comprises a number of segments 22 each of which is made of an endless metal loop that has been bent into a plurality of straight sections or struts 24 that are integrally joined by discrete axial turns, or crowns 26. Axially adjacent segments 22 may be joined to one another at one or more of their crowns 26. These connections may be made by welding, soldering, adhesive bonding, mechanical fastening, or in any other suitable manner. The pattern of the segments 22 can be W-shaped or can be a more complex shape with the elements of one segment continuing into the adjacent segment. Each segment 22 may have more undulations than are shown in FIG. 2. FIG. 3 depicts yet another exemplary stent appropriate for use with embodiments disclosed herein.

As is understood by one of ordinary skill in the art, stents used in various embodiments disclosed herein can be pretreated prior to applying the biological scaffold seeded with endothelial cells. The pretreatment can include chemical etching. The pretreatment can also or alternatively include plasma etching to generate a thick passive oxide layer on the metal surface that improves corrosion resistance against biological fluids. Pretreatment can also make the surface microstructure rougher and improve adhesion of the biological scaffold.

### Biological Scaffolds

Suitable biological scaffolds for use with the embodiments disclosed herein include, without limitation, extracellular matrix material, submucosa, dura mater, pericardium, serosa, peritoneum, basement membrane tissue, liver basement membrane, intestinal submucosa, small intestinal submucosa, stomach submucosa, urinary bladder submucosa, and uterine submucosa. These biological scaffold materials can be derived generally from warm-blooded vertebrates including, without limitation, porcine, bovine or ovine mammals. Human donor tissues may also be used. These biological scaffold materials may be used in any suitable form, including as layers.

The biological scaffold material used can be purified and sterilized in any suitable manner. Exemplary purification processes can involve contacting the material with an appropriate agent or agents. For example, biological scaffolds can be tanned with glutaraldehyde and formaldehyde, treated with oxidizing compounds or subjected to gas plasma sterilization, gamma radiation, or combinations thereof. In this regard, appropriate processes involve exposing the isolated biological scaffold to a solution containing one or more oxidizing agents, peroxy compounds, organic peroxy compounds, or peracids. When a peracid is used, it can be selected from the group consisting of peracetic acid, perpropionic acid and perbenzoic acid. Other peroxy disinfecting agents, such as hydrogen peroxide, can also be used. Still other suitable peroxy compounds are described in “Peroxygen Compounds”, S. Block, in Disinfection, Sterilization and Preservation, S. Block, Editor, 4th Edition, Philadelphia, Lea & Febiger, pp. 167-181, 1991; and “Disinfection with peroxygens” M. G. C. Baldry and J. A. L. Fraser, in Industrial Biocides, K. Payne, Editor, New York, John Wiley and Sons, pp. 91-116, 1988, which are incorporated by reference herein for their discussion of the same. Other oxidizing agents, for example, chlorine agents such as chlorhexidine (1,6-di(4-chlorophenyldiguanido)hex-ane) in its digluconate form can also be used. Many other suitable chlorine agents are described in “Chlorhexidine”, G. W. Denton, in Disinfection, Sterilization and Preservation, S. Block, Editor, 4th Edition, Philadelphia, Lea & Febiger, pp. 274-289, 1991, which is incorporated by reference herein for its discussion of the same.

Appropriate solvents for diluting the oxidizing agent are aqueous alcohols. Alcohol content can be from about 1% to about 30% by volume of the solution in one embodiment, and from about 2% to about 10% by volume in another embodiment. Many alcohols can be used to form the aqueous alcohol solution. In certain embodiment, the alcohol contains from 1 to about 6 carbon atoms. The alcohol can also be selected from the group consisting of ethanol, propanol, isopropanol, denatured alcohol or butanol. In addition, the solution generally has a pH of about 1.5 to about 10, about 2 to about 6, or about 2 to about 4. Although not necessary, conventional buffers can be used to adjust the pH.

The biological scaffold material can be exposed to the above-described processing agents for a suitable period of time. Generally, exposure can entail submersing the isolated material into a solution under agitation. The exposure time is typically at least about 5 minutes, and often in the range of about 15 minutes to about 40 hours, and more typically in the range of about 0.5 hours to about 8 hours. In one embodiment, the biological scaffold material can be pre-rinsed with a solvent, for example sterile water, before exposure to the processing solution.

One exemplary purification procedure involves exposing the biological scaffold material to dilute peracetic acid. The peracetic acid is diluted with an aqueous alcohol solution containing about 2% to about 10% by volume alcohol. The concentration of the peracetic acid can range, for example, from about 0.05% by volume to about 1.0% by volume. When the peracetic acid content is about 0.2%, the biological scaffold material can be exposed for about two hours. The exposure time can, of course, be longer or shorter, depending upon the particular agent used, its concentration, and other factors within the purview of those of ordinary skill in the art.

In one embodiment, small intestinal submucosa can be harvested and prepared for use as a biological scaffold as described in U.S. Pat. No. 6,206,931 which is incorporated by reference herein for its discussion of the same. This small intestinal submucosa can be formed into a single- or multi-layer tube using the techniques described in any one of U.S. Pat. Nos. 6,187,039, 6,206,931 and 6,358,284, and in WO 0110355 published Feb. 15, 2001 which are all incorporated by reference herein for their discussions of the same.

When the biological scaffold material used is submucosa, for example small intestinal submucosa, the source tissue can be disinfected prior to harvesting the submucosa. Suitable procedures are disclosed, for example, in U.S. Pat. No. 6,206,931 which is incorporated by reference herein for its discussion of the same. Biological scaffold materials, including submucosa materials, can also be processed to retain one or more bioactive components with which they occur. These may include, for example, one or more growth factors such as basic fibroblast growth factor (FGF-2), transforming growth factor beta (TGF-beta), epidermal growth factor (EGF), and/or platelet derived growth factor (PDGF). Biological scaffold material can also include other biological materials such as heparin, heparin sulfate, hyaluronic acid, fibronectin and the like.

The described tissue processing techniques can be used to not only remove cell and cell debris, but also possible endogenous viruses, prion agents, and any contaminants introduced during harvesting of the biological scaffold material. Illustratively, prion inactivation can be undertaken using sodium hydroxide treatment. Suitably, the material can be contacted with a solution of sodium hydroxide for a period of time sufficient to inactivate any prions present. The duration of contact will vary with the concentration of the sodium hydroxide solution, and potentially other factors known to those of ordinary skill in the art. Illustratively, the tissue material can be contacted with an about 0.1 N sodium hydroxide solution for about 5 minutes to about 5 hours, for about 10 minutes to about 2 hours, or for about 15 to about 60 minutes. Alternatively, more concentrated solutions of sodium hydroxide can be used, e.g. by contacting the tissue with about 1.0 N sodium hydroxide for about 15 to about 60 minutes. Still other prion inactivation treatments are known and can be used, including for example the use of steam sterilization under pressure, contact with a sodium hypochlorite solution (e.g. about 2.5%), and the like.

### Endothelial Cells

Endothelial cells suitable for seeding onto the biological scaffolds disclosed herein (or the precursors thereto) can be derived from any suitable source of such cells including vascular endothelial cells from arterial or venous tissues. The cells for the tissue graft may be an autograft, allograft, biograft, biogenic graft or xenograft. The cells may be autologous to a patient to be treated, allogenic to the patient to be treated, or xenogenic to the patient to be treated. The cells may be derived and potentially expanded from biopsy tissue, or may be derived from stable cell lines, including human cell lines.

The chosen cells generally will be disaggregated from an appropriate organ or tissue. This disaggregation may be readily accomplished using techniques known to those of ordinary skill in the art. Such techniques include disaggregation through the use of mechanical forces either alone or in combination with digestive enzymes and/or chelating agents that weaken cell-cell connections between neighboring cells to make it possible to disperse the tissue into a suspension of individual cells without appreciable cell breakage. Enzymatic dissociation can be accomplished by mincing the tissue and treating the minced tissue with any of a number of digestive enzymes either alone or in combination. Digestive enzymes include but are not limited to trypsin, chymotrypsin, collagenase, elastase, and/or hyaluronidase, Dnase, pronase, etc. Mechanical disruption can also be accomplished by a number of methods including, but not limited to the use of grinders, blenders, sieves, homogenizers, pressure cells, or sonicators. For a review of tissue disaggregation techniques, see Freshney, Culture of Animal Cells. A Manual of Basic Technique, 2d Ed., A. R. Liss, Inc., New York, 1987, Ch. 9, pp. 107-126 which is incorporated by reference herein for its discussion of the same.

Once the primary cells are disaggregated, the cells can be separated into individual cell types using techniques known to those of ordinary skill in the art. For a review of clonal selection and cell separation techniques, see Freshney, Culture of Animal Cells. A Manual of Basic Techniques, 2d Ed., A. R. Liss, Inc., New York, 1987, Ch. 11 and 12, pp. 137-168, which is incorporated by reference herein for its discussion of the same. Media and buffer conditions for growth of the cells will depend on the type of cell and such conditions are known to those of ordinary skill in the art.

In certain embodiments, the cells can be grown in bioreactors. A bioreactor may be of any class, size or have any one or number of desired features, depending on the product to be achieved. Different types of bioreactors include tank bioreactors, immobilized cell bioreactors, hollow fiber and membrane bioreactors, as well as digesters. Three classes of immobilized bioreactors allow cell growth: membrane bioreactors, filter or mesh bioreactors, and carrier particle systems. Membrane bioreactors grow the cells on or behind a permeable membrane, allowing the nutrients to leave the cell, while preventing the cells from escaping. Filter or mesh bioreactors grow the cells on an open mesh of an inert material, allowing the culture medium to flow past, while preventing the cells from escaping. Carrier particle systems grow the cells on something very small, such as small nylon or gelatin beads. The bioreactor can be a fluidized bed or a solid bed. Other types of bioreactors include pond reactors and tower fermentors.

Endothelial cells seeded onto the biological scaffolds disclosed herein can be genetically engineered to express a biologically active or therapeutically effective protein product. Techniques for modifying cells to produce the recombinant expression of such protein products are well known to those of ordinary skill in the art.

Biological Scaffold Seeded with Endothelial Cells

The biological scaffold seeded with endothelial cells can be coated on the stent before the stent is placed in the artery, or can be grown after arterial placement by several factors that encourage such growth.

In certain embodiments, the biological scaffold seeded with endothelial cells can be about 1 to about 3 cells thick. In another embodiment, the biological scaffold seeded with endothelial cells can be formed of one or more layers of extracellular matrix material, for example including one to about four or more layers of extracellular matrix material. These layers can be bonded to another by any suitable method, including, without limitation, the use of biocompatible adhesives such as collagen pastes, fibrin glue, and the like. Layers can also be dehydrothermally bonded to one another, for example by compressing overlapped regions under dehydrating conditions.

The biological scaffold material can be configured to a tubular form either before or after the cells are seeded. For example, in certain embodiments, the cells are provided on the biological scaffold material while the same is in a sheet configuration, and the sheet is thereafter configured to a tube, e.g. after a period of culturing in vitro. In other embodiments, the biological scaffold material can be configured to a tube, and then cells are provided and potentially cultured for a period in vitro on the same. Further, some cells may be added while the biological scaffold material is in sheet form, and others after configuration to a tube. For instance, cells to populate in the interior lumen of the stent construct can be added and potentially cultured with the biological scaffold material in sheet form, the sheet form then being configured to a tube form, and additional cells then being added to the interior and/or exterior surfaces of the tube construct.

When adding and culturing cells with the biological scaffold material in tube form, it can be beneficial in some instances to provide a tubular support or other means to retain the material in its tube form as the cells are cultured, and to prevent any undesired bridging of cells across the interior lumen that may cause a deleterious blockage.

To prepare tubular graft constructs, flat sheet biological scaffold materials can be configured to a tubular form in any suitable manner. These include, for example, techniques in which a flat sheet of biological scaffold material is configured into a tube shape, and sutured or otherwise bonded to retain the tube shape. Suitable methods for forming tubes of collagen tissues are disclosed in U.S. Pat. Nos. 6,187,039, 6,206,931 and 6,358,284, and in WO 0110355 published Feb. 15, 2001 which are incorporated by reference herein for their discussions of the same.

### Structure

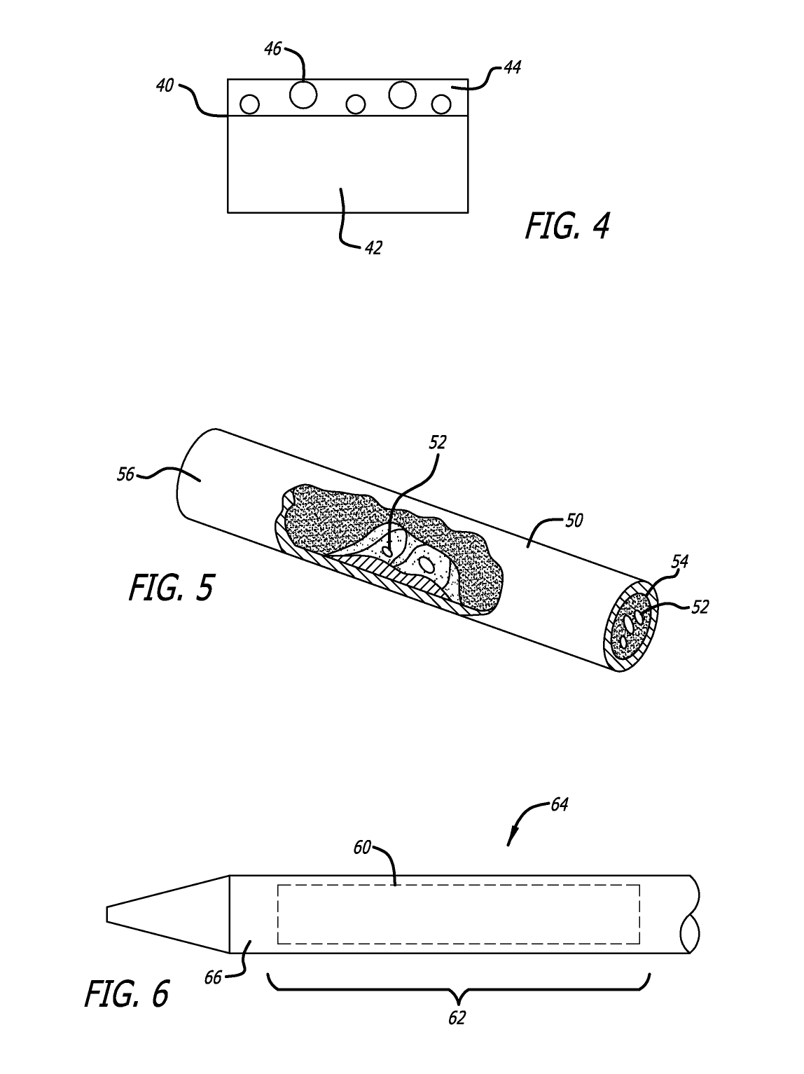

As will be understood by one of ordinary skill in the art, the biological scaffold seeded with endothelial cells can be seeded on the interior (lumenal) surface, the exterior surface, or both surfaces of the stent. FIG. 4 depicts an embodiment wherein one surface 40 of a stent strut 42 is coated with the biological scaffold 44 seeded with endothelial cells 46. In this embodiment, the biocompatible scaffold 44 is illustrated as disposed on an inner surface 40 of the stent strut 42. FIG. 5 depicts a perspective view of an embodiment wherein both stent surfaces are coated with the biocompatible biological scaffold 50 seeded with endothelial cells 52 only on the inner surface 54 of the stent construct 56.

For delivery, the stents disclosed herein can be loaded into a delivery catheter such as that depicted in FIG. 6. The stent 60 is radially compressed to fill the stent chamber 62 in the distal end of delivery catheter 64. The stent 60 is covered with a retractable sheath 66 that is retracted at the implantation site to allow deployment of the stent.

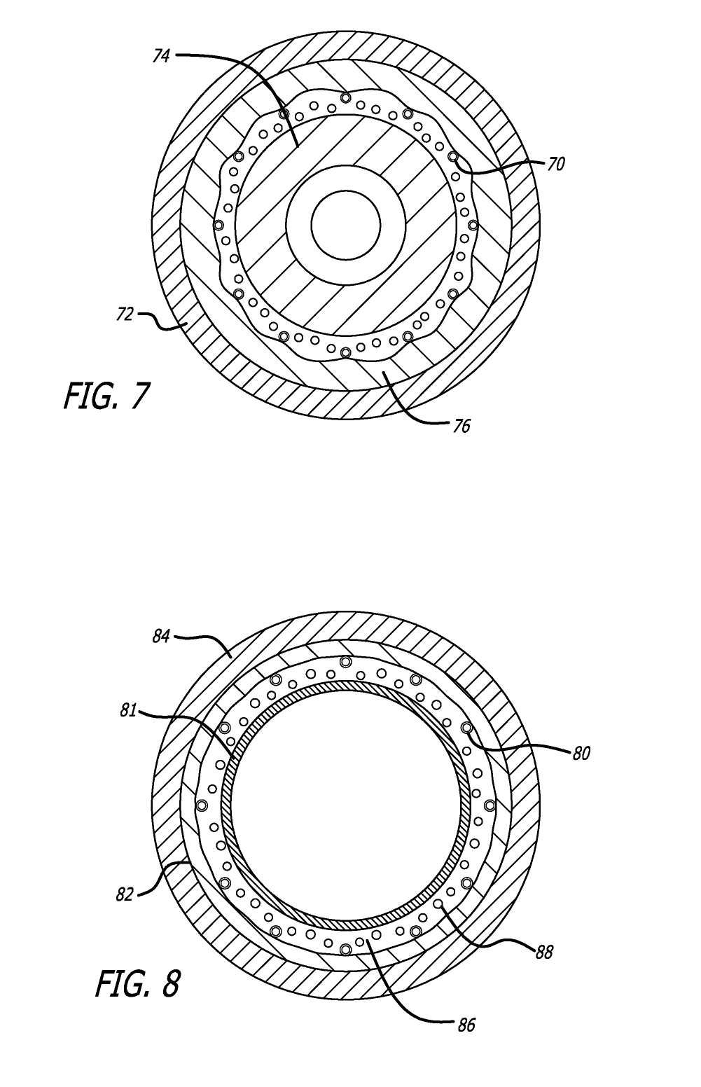

FIG. 7 shows a stent 70 as it would appear in cross-section in an artery 72 occluded with plaque 76 with an angioplasty balloon 74 expanded in position within the stent 70.

FIG. 8 shows the stent 80 deployed after the angioplasty balloon has been removed. The stent 80 has been expanded against the inner wall 82 of the occluded artery 84 as the angioplasty balloon was expanded. The biological scaffold 86 seeded with endothelial cells 88 provides a suitable surface for presence and growth of an endothelial cell lining 81.

### Additional Bioactive Materials

In addition to endothelial cells, the biological scaffold may be seeded with a therapeutic agent such as, without limitation, anti-inflammatory agents, NSAIDS, selective COX-2 enzyme inhibitors, antibacterial agents, antiparasitic agents, antifungal agents antiviricides, antiviral agents, analgesic agents, antisense nucleotides, thrombin inhibitors, antithrombogenic agents, tissue plasminogen activators, thrombolytic agents, fibrinolytic agents, vasospasm inhibitors, calcium channel blockers, nitrates, nitric oxide promoters, vasodilators, antimicrobial agents, antibiotics, antiplatelet agents, antimitotics, microtubule inhibitors, actin inhibitors, remodeling inhibitors, agents for molecular genetic intervention, cell cycle inhibitors, inhibitors of the surface glycoprotein receptor, antimetabolites, antiproliferative agents, anti-cancer chemotherapeutic agents, anti-inflammatory steroids, immunosuppressive agents, radiotherapeutic agents, iodine-containing compounds, barium-containing compounds, heavy metals functioning as a radiopaque agent, peptides, proteins, enzymes, biologic agents, angiotensin converting enzyme (ACE) inhibitors, ascorbic acid, free radical scavengers, iron chelators, antioxidants, a radiolabelled form of any of the foregoing, or a combination or mixture of any of these.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A stent comprising a biological scaffold seeded with endothelial cells on the inner surfaces of said stent.

2. A stent of claim 1 wherein said biological scaffold is also on the outer surfaces of said stent.

3. A stent of claim 1 wherein said biological scaffold comprises extracellular matrix material, submucosa, dura mater, pericardium, serosa, peritoneum and/or a basement membrane tissue.

4. A stent of claim 3 wherein said submucosa comprises intestinal submucosa, stomach submucosa, urinary bladder submucosa and/or uterine submucosa.

5. A stent of claim 3 wherein said submucosa comprises at least one growth factor.

6. A stent of claim 5 wherein said at least one growth factor is basic fibroblast growth factor, transforming growth factor beta, epidermal growth factor and/or platelet derived growth factor.

7. A stent of claim 3 wherein said basement membrane tissue comprises liver basement membrane tissue.

8. A stent of claim 1 wherein said biological scaffold is derived from a warm-blooded vertebrate.

9. A stent of claim 1 wherein said biological scaffold comprises a therapeutic agent.

10. A stent of claim 1 wherein said endothelial cells are arterial and/or venous vascular endothelial cells.

11. A stent of claim 1 wherein said endothelial cells are genetically engineered to express a biologically active protein product.

12. A stent of claim 1 wherein said biological scaffold seeded with endothelial cells is about 1 to about 3 cells thick.

13. A method of forming a stent comprising a biological scaffold seeded with endothelial cells comprising:
providing a stent;
seeding a biological scaffold with endothelial cells; and
associating said biological scaffold seeded with said endothelial cells with said stent.

14. A method of claim 13 further comprising:
treating said biological scaffold with glutaraldehyde, formaldehyde, oxidizing compounds, gas plasma sterilization and/or gamma radiation.

15. A method of claim 14 wherein said oxidizing compound is a peracid diluted in alcohol.

16. A method of claim 15 wherein said peracid is peracetic acid, perpropionic acid or prebenzoic acid and wherein said alcohol is ethanol, propanol, isopropanol, dentatured alcohol or butanol.

17. A method of claim 14 further comprising:
pre-rinsing said biological scaffold with a sterile solvent before said treating.

18. A method of claim 13 wherein said biological scaffold is submucosa and said method further comprises:
processing said submucosa to retain at least one growth factor.

19. A method of claim 18 wherein said at least one growth factor is basic fibroblast growth factor, transforming growth factor beta, epidermal growth factor and/or platelet derived growth factor.

20. A method of claim 13 wherein said method further comprises:
genetically engineering said endothelial cells to express a biologically active protein product.
